(19) United States
(12) Patent Application Publication
(10) Pub. No.: US 2019/0368976 A1
(43) Pub. Date: Dec. 5, 2019

(54) WIPE FOR EXPLOSIVES AND NARCOTICS DETECTION

(71) Applicant: Illinois Tool Works Inc., Glenview, IL (US)

(72) Inventor: Paul M. Blair, Jonesboro, GA (US)

(21) Appl. No.: 16/419,217

(22) Filed: May 22, 2019

Related U.S. Application Data
(60) Provisional application No. 62/680,874, filed on Jun. 5, 2018.

(51) Int. Cl. G01N 1/02; G01N 21/84; G01N 21/77; G01N 31/22; G01N 33/22

(52) U.S. Cl. CPC G01N 1/02; G01N 21/84; G01N 21/77; G01N 2001/028; G01N 33/227; G01N 2001/022; G01N 31/22

(57) ABSTRACT

A wipe for collecting a sample from a surface for analysis in a detector includes a substrate having first and second surfaces. The wipe can have a high-heat tolerant polymeric material on the substrate and a plurality of openings formed in the substrate and polymeric material to form the wipe. The wipe is flexible and conforms to the surface for collecting the sample. The wipe adsorbs the sample from the surface and desorbs the sample in the detector. The wipe is reusable for collecting one or more subsequent samples.

### CROSS-REFERENCE TO RELATED APPLICATION DATA

This application claims the benefit of and priority to Provisional U.S. Patent Application Ser. No. 62/680,874, filed Jun. 5, 2018, titled, WIPE FOR EXPLOSIVES AND NARCOTICS DETECTION, the disclosure of which is incorporated herein in its entirety.

### BACKGROUND

The present disclosure pertains to a wipe for collecting a sample from a surface for analysis in a detector, a method of manufacturing the wipe and a method of collecting samples using the wipe. More particularly, the present disclosure pertains to a wipe for collecting a sample of, for example, explosives and narcotics compounds, for detection by various analytical means, such as an ion mobility spectrometer (IMS), an ion trap mobility spectrometer (ITMS), a mass spectrometer (MS), a non-linear dependence of ion mobility (NLDM), and other methods, the compounds being present either as traces within particles or as discrete particles or aerosols, droplets or the like.

Finding traces of explosives, narcotics, and other contraband helps security authorities locate hidden contraband in transported goods and cargo, or in vehicles and aircraft. However, embodiments consistent with the present disclosure also encompass collecting samples on a DNA sample. Known wipes for collecting samples in the prior art have typically been made of paper, cotton cloth, glass fibers coated in polytetrafluoroethylene (PTFE/Teflon), or aromatic polyamide (Nomex) fabric.

Each of these prior art sampling media have their own shortcomings. For example, paper and cotton wipes may pick up particles efficiently but also absorb water. The water requires evaporation, and as such there is a delay in processing the target materials which can inhibit the detection process. Moreover, often, paper and cotton wipes are not reusable.

PTFE-coated fiberglass or aromatic polyamide fabric may be reusable. However, these materials have a lower coefficient of friction, and thus do not efficiently remove particles from rough surfaces.

Accordingly, there exists a need for a wipe for collecting a sample from a surface for analysis in a detector, which wipe is made from a non-water-absorbent material that effectively absorbs or collects target material particles from a variety of surfaces. The is also a need for a method of manufacturing such a wipe and a method of collecting target materials. More desirably still, such a material is more adsorbent and more reusable than known wipes.

### SUMMARY

A wipe for collecting a sample from a surface for analysis in a detector, a method of manufacturing such a wipe and a method of collecting a sample of target material using such a wipe are disclosed. In an embodiment, the wipe is formed from a mesh. In an embodiment the mesh is a metal wire mesh. The mesh is flexible and conforms to the surface for collecting the sample, adsorbs the sample from the surface at a first temperature range, and desorbs the sample in the detector at a second temperature. In an embodiment, the wipe is reusable for collecting a plurality of samples.

In a presently contemplated wipe, the mesh is a plain weave of the metal wire. Optionally, the mesh can be a Dutch weave, a twilled weave, a twilled Dutch weave, and a reverse Dutch weave of the metal wire, or other forms of metal such as a perforated metal sheet, or an expanded metal sheet. A variety of mesh counts are possible, such as 200 per inch, 400 per inch, and 400×1400 per square inch. Similarly, a variety of metal wire diameters are possible, such as 0.001″, 0.0016″, 0.002″, 0.0021″, and 0.0028″. Additionally, the wipe can further include a textured surface of the metal wire, a coating on the metal wire, and/or at least one aperture in the mesh for attaching the wipe to a collection tool.

In an embodiment, a wipe for collecting a sample from a surface for analysis in a detector includes a substrate having first and second surfaces, a high-heat tolerant polymeric material on the substrate and a plurality of openings formed in the substrate and polymeric material to form the wipe. The wipe is flexible and conforms to the surface for collecting the sample. The wipe adsorbs the sample from the surface and desorbs the sample in the detector. Such a wipe is reusable for collecting one or more subsequent samples.

In an embodiment the substrate is a metal. In an embodiment, the substrate is a polymeric material. The polymeric material is one or a combination of polyether ether ketone (PEEK), polyimide and silicone. The substrate is formed as a perforated foil and the polymeric material is applied to the perforated foil. The polymeric material can be present on one or both of the first and second sides.

A method for manufacturing the wipe includes cutting the wipe from a sheet of material, wherein the wipe is flexible and conforms to a surface for collecting the sample, cleaning the wipe to remove contamination and packaging the wipe in a clean container. The method can include providing a substrate having first and second sides and applying a high-heat tolerant polymeric material on the substrate. The high-heat tolerant polymeric material can be applied to one or both of the first and second sides of the substrate. In an embodiment the substrate is formed from a metal.

A method of manufacturing the wipe can include cutting the wipe from a sheet of metal, cleaning the wipe to remove contamination, and packaging the wipe in a clean or sterile container. The sheet of metal is, for example, a mesh, a weave of metal wire, a perforated metal foil, or an expanded metal foil.

In a presently contemplated method, the wipe is cleaned by heating the wipe and/or soaking the wipe in, for example, an acid or a solvent. A variety of sheets of metal may be used, such as a plain weave of a metal wire, a perforated metal foil, or an expanded metal foil. Similarly, a variety of cutting techniques may be used, such as die cutting, laser cutting, and waterjet cutting. Additionally, the method can further include texturing a surface of the wipe, applying a coating to the wipe, and/or cutting at least one aperture in the wipe. Furthermore, the method can further include fusing edges of the wipe using a high energy or high pressure process to reduce fraying of the edges.

These and other features and advantages of the present device will be apparent from the following description, taken in conjunction with the accompanying sheets of drawings, and in conjunction with the appended claims.

### BRIEF DESCRIPTION OF THE DRAWINGS

The benefits and advantages of the present disclosure will become more readily apparent to those of ordinary skill in the relevant art after reviewing the following detailed description and accompanying drawings, wherein:

FIG. 1 is a schematic view of an example of a known detector;

FIG. 2 is a top view of a prior art wipe for collecting a sample from a surface for analysis in the detector;

FIGS. 2A-D are top views of embodiments of a variety of wipes for collecting a sample from a surface for analysis in the detector;

FIG. 3 is a top view of a metal foil/mesh from which the variety of wipes can be manufactured; and

FIG. 4 is a flowchart of an exemplary process of manufacturing the wipes.

### DETAILED DESCRIPTION

While the present disclosure is susceptible of embodiment in various forms, there is shown in the drawings and will hereinafter be described one or more presently preferred embodiments with the understanding that the present disclosure is to be considered an exemplification of the present devices and methods and is not intended to limit the disclosure to the specific embodiments illustrated.

It should be further understood that the title of this section of this specification, namely, “Detailed Description”, relates to a requirement of the United States Patent Office, and does not imply, nor should be inferred to limit the subject matter disclosed herein.

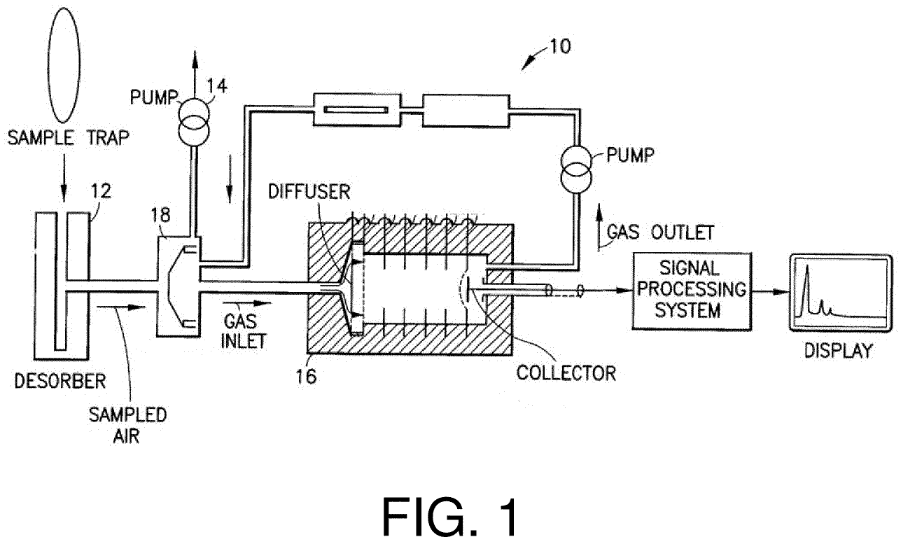

An existing detector 10 of explosives and narcotics is illustrated schematically in FIG. 1. The detector 10 of FIG. 1 analyzes samples that are collected on sample wipes. The wipes (i.e., sample traps) are positioned in a thermal desorber 12. The thermal desorber is heated to a temperature, such as, without limitation, 55° C., 100° C., or 200° C., which temperature is greater than the adsorption temperature range of the wipe. Desorbed material is carried into the detector by the action of a sampling pump 14. Sample air is drawn into an ionization chamber 16 over a membrane 18. Some contraband or other materials of interest diffuse through the membrane 18 and into the chamber 16, which may be an ion mobility spectrometer (IMS), an ion trap mobility spectrometer (ITMS), a mass spectrometer (MS), or a non-linear dependence of ion mobility (NLDM) or like detector. The membrane 18 eliminates dust and most atmospheric materials, including, for example, water, all of which may affect proper functioning of the detector 10. Unfortunately, the membrane 18 is minimally efficient at transferring the materials of interest, and this efficiency can limit the ultimate sensitivity of the detector 10.

Some of the manufacturers and models of explosives and narcotics trace detectors include:
SAFRAN Morpho: Itemiser, MobileTrace
Smiths Detection: IONSCAN, SABRE, TRACE-PRO, MMTD
L3 SDS: OptEX, B220, H150
Rapiscan Systems: DETECTRA
FLIR Systems: Fido, Griffin
Sibel Ltd.: MO-2, MO-8
GE Ion Track: Itemiser, VaporTracer
Implant Sciences: B220, H150

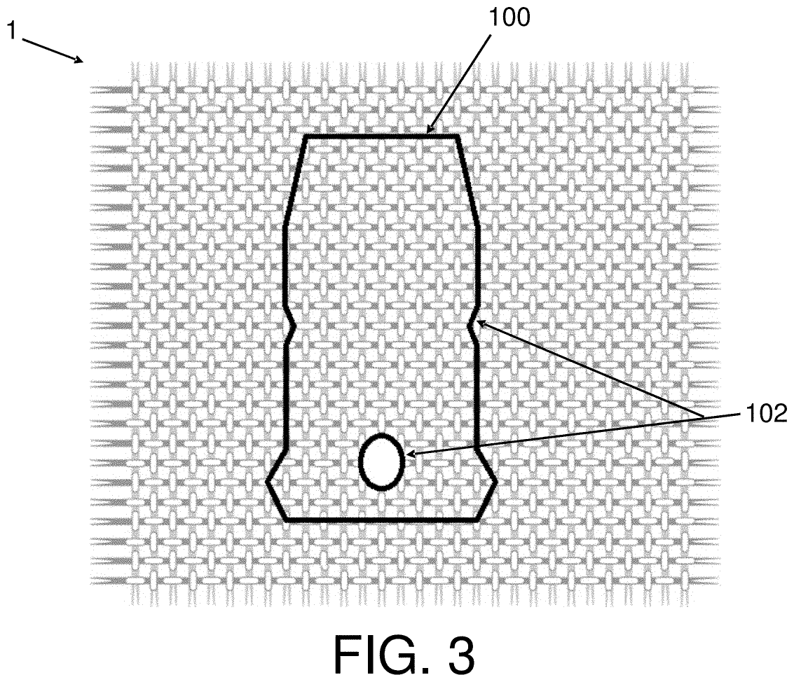

Referring to FIG. 3, in view of the above, an embodiment consistent with the present disclosure provides a wipe 100 formed from a mesh 1 of metal wire that is efficiently adsorbent and desorbent, and is reusable for collecting a plurality of samples for repeated analysis in the detector 10. Metal is one desirable material for the wipe 100 because it is a ductile material that can be finely drawn and woven and will withstand repeated uses. Metals such as copper, aluminum, and stainless steel have good thermal conductivity and corrosion resistance. Thin or fine metal wires in a woven mesh pattern are flexible, conforming to a variety of surfaces for sample collection. In addition, such mesh patterns have many open areas and as such, increased surface area for adsorption of samples. Moreover, such mesh patterns can be formed having a soft feel for wiping skin and/or personal items without injury or damage. A combination of these materials metals, or a metal/alloy that will be recognized by those skilled in the art may be suitable for the wipe 100.

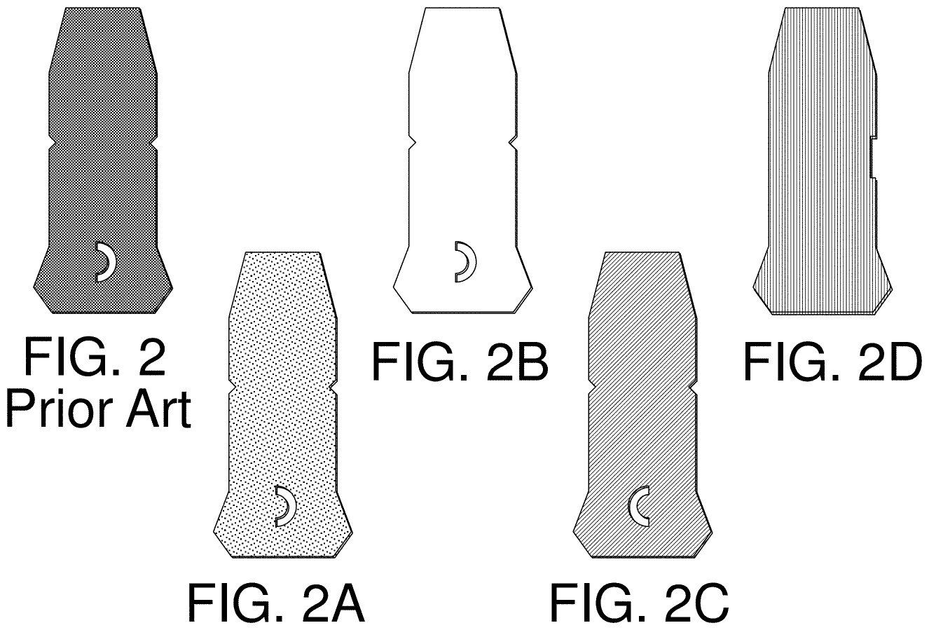

FIGS. 2A-D are top views of a variety of wipes 100 for collecting a sample from a surface for analysis in the detector consistent with the present disclosure. Wipes are also known in the art as, for example, but not limited to, “swabs” and “traps.” The wipes 100A-D may be of a variety of shapes. FIGS. 2A-2D illustrate an irregular polygon, but those skilled in the art will understand that other shapes are possible, such as, but not limited to, rectangles and ovals and the like. A prior art wipe (PTFE-coated fiberglass) is shown in FIG. 2 and labeled “Prior Art” for comparison.

FIG. 2A depicts an illustrative example of a wipe 100A formed from an aluminum mesh. A count of the mesh is 200 aluminum wires per inch in both the shute and warp directions. The warp direction is that direction along the length of the mesh as formed and the shute or weft direction is that direction across the width of the mesh as formed. Wires are also known in the art as, for example, but not limited to, “strands” and “filaments.” A diameter of the aluminum wire is 0.0021″. Other combinations of mesh count and wire diameter, such as those described with respect to FIGS. 2B-D, are possible in additional embodiments consistent with the present disclosure.

FIG. 2B depicts an illustrative example of a wipe 100B formed from a stainless steel mesh. The count of the mesh is 400 stainless steel wires per inch in both the shute and warp directions, and the diameter of the stainless steel wire is 0.001″. Other combinations of mesh count and wire diameter, such as those described with respect to FIGS. 2A and 2C-D, are possible in additional embodiments consistent with the present disclosure.

FIG. 2C depicts an illustrative example of a wipe 100C formed from a copper mesh. The count of the mesh is 200 copper wires per inch in both the shute and warp directions. The diameter of the copper wire is 0.002″. Other combinations of mesh count and wire diameter, such as those described with respect to FIGS. 2A-B and 2D, are possible in additional embodiments consistent with the present disclosure.

FIG. 2D depicts an illustrative example of a wipe 100D formed from a stainless steel mesh. The count of the mesh is 400 stainless steel wires per inch in the shute direction and 1400 stainless steel wires per inch in the warp direction, described alternately as 400×1400 per square inch. The diameter of the stainless steel wire is 0.0028″ in the shute direction and 0.0016″ in the warp direction.

Wipes 100A-D formed from a mesh of metal wire have a large surface area for adsorption of samples from surfaces. The structure of the mesh (described in more detail with respect to FIG. 3) is able to capture and hold samples, then desorb (release) samples into the detector 10 during heating. The thin wire diameter and the thermal conductivity of metals of wipes 100A-D allow for rapid and even heat-up of the wipes 100A-D. The rapid and even heat-up improves the speed of the detector 10 completing an analysis. An even heat-up also produces a more complete desorption of the collected sample, improving the accuracy of the detector 10 analysis and preparing the wipes 100A-D for reuse in collecting additional samples. It is contemplated that the wipes 100A-D may be reused, for example, 12-15 times.

Explosives which can be collected in a sample using wipes 100A-D include, without limitation, organic traces (nitroaromatics, cyclonitroaliphatics) and inorganic traces (nitrates, perchlorates). More broadly, explosives which can collected in a sample using wipes 100A-D include, but are not limited to, 2-amino-4,6-dinitrotoluene, 4-amino-2,6-dinitrotoluene, ammonal, ammonium nitrate, black powder, 2,4-dimethyl-1,3-dinitrobutane, 2,4-dinitrotoluene, ethylene glycol dinitrate, forcite 40, GOMA-2, hexanitrostilbene, 1,3,5,7-tetranitro-1,3,5,7-tetrazacyclooctane (HMX), mononitrotoluene, nitroglycerine, pentaerythritol tetranitrate (PETN), 1,3,5-trinitro-1,3,5-triazacyclohexane (RDX), semtex-A, Semtex-H, smokeless powder, trinitro-2,4,6-phenylmethylnitramine tetryl (Tetryl), 2,4,6-trinitrotoluene (TNT), trilita, 1,3,5-trinitrobenzene, and combinations of these compounds.

Narcotics which can be collected in a sample using wipes 100A-D include, but are not limited to 6-acetylmorphine, alprazolam, amobarbital, amphetamine, antipyrine, benzocaine, benzoylecgonine, bromazepam, butalbital, carbetapentane, cathinone, chloradiazepoxide, chlorpheniramine, cocaethylene, cocaine, codeine, diazepam, ecgonine, ecognine methyl ester (EME), ephedrine, fentanyl, flunitrazepam, hashish, heroin, hydrocodone, hydromorphone, ketamine, lidocaine, lorazepam, lysergic acid diethylamide (LSD), lysergic acid, N-methyl-1-3(3,4-methylenedioxyohenyl)-2-butanamine (MBDB), 3,4-methylenedioxyamphetamine (MDA), DL-3,4-methyl enedioxyethylamphetamine (MDEA), methylenedioxymethamphetamine (MDMA), marijuana, mescaline, methadone, methamphetamine, methaqualone, methcathinone, morphine, noscapine, opium, oxazepam, oxycodone, phencyclidine (PCP), pentobarbital, phenobarbital, procaine, psilocybin, secobarbital, temazepam, THC, THC—COOH, triazolam, and combinations of these compounds.

Furthermore, in addition to other detectable materials, it is possible for the wipes 100A-D to collect a DNA sample for use by other detectors. The flexibility, adsorption, and desorption of the wipes 100A-D allows DNA samples to be collected from the human body since metals (e.g., copper, aluminum, stainless steel, a combination of the listed metals, or a metal/alloy known in the art) are safe for human contact.

FIG. 3 is a top view of an illustrative metal foil or mesh 1 from which a wipe 100 consistent with the present disclosure may be are manufactured. As described previously, mesh 1 may be formed from a plurality of metal wires. The mesh 1 may be formed by weaving the wires together along a shute direction and along a warp direction. Other methods of forming the mesh, for example, but not limited to, knitting, braiding, and tangling, are possible.

The illustrative mesh 1 of FIG. 3 is a plain weave pattern, also known as a 1×1 twill pattern. An equal number of shute wires and warp wires are present in an inch of mesh 1. In other embodiments, the mesh may be, for example, a Dutch weave, a twilled weave, a twilled Dutch weave, a reverse Dutch weave pattern, and the like.

Characteristics of the mesh 1—adsorption, desorption, heat tolerance, and porosity—can be changed by adjusting the mesh count, the wire diameter, or the weave pattern, which in turn may vary the surface area of the wipe. As described above with respect to FIGS. 2A-D, a variety of mesh counts and wire diameters may be used. An open area between metal wires in the mesh is a function of the mesh count, wire diameter, and the weave pattern. The open area is typically measured in microns for wipes used in explosives and narcotics detection. For example, a 200 mesh count wipe has openings of approximately 74 microns, a 400 mesh count wipe has openings of approximately 38 microns, and a 400×1400 mesh count wipe has openings of approximately 5-6 microns.

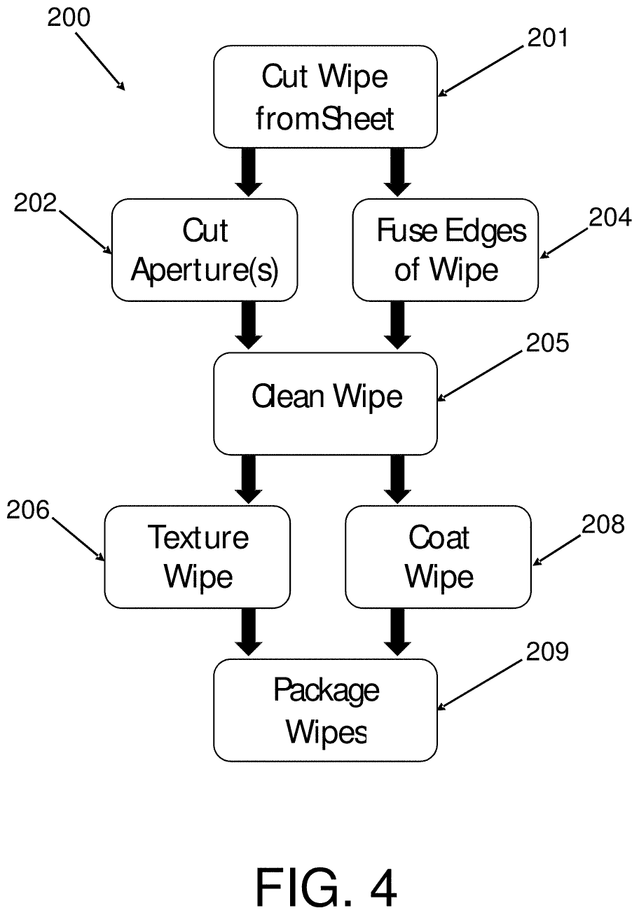

The wipe 100 is typically used in room temperature settings or typical outdoor temperatures. In one embodiment, the wipe 100 is able to adsorb a sample from a surface at a first temperature (i.e., an adsorption temperature range) of about −10° C. to about 55° C. In another embodiment, the wipe 100 is able to adsorb the sample at a first temperature range of about 0° C. to about 30° C. In yet another embodiment, the wipe 100 is able to adsorb the sample at a first temperature range of about 10° C. to about 20° C. The sample is desorbed in the detector 10 by heating and airflow. In one embodiment, the wipe 100 desorbs the sample at a second temperature greater than about 100° C. In another embodiment, the wipe 100 desorbs the sample at a second temperature greater than about 200° C. In yet another embodiment, the wipe 100 desorbs the sample at a second temperature greater than about 55° C. Higher temperatures, such as those greater than about 200° C., may also be used to clean the wipe 100 during manufacture (described in more detail with respect to FIG. 4).

The wipe 100 of FIG. 3 includes at least one aperture 102. In one embodiment, the at least one aperture 102 allows for attaching the wipe 100 to a collection tool. Collection tools are typically used for explosives and trace detection to reduce handling of the wipe 100 and improve analysis accuracy. The at least one aperture 102 may also be used to secure the wipe 100 to the detector 10. In another embodiment, the wipe 100 is configured to attach to a collection tool or the detector 10 without an aperture. For example, without limitation, a portion of the wipe 100 is clasped between components of a collection tool or the detector 10.

Edges of the wipe 100, formed from cutting the wipe 100 from the foil of mesh 1 or cutting the at least one aperture 102 from the wipe 100, may be treated to reduce or eliminate fraying. In one embodiment, the metal wires on the edge of the wipe 100 are fused, also known as sealed, using a high-energy process. In another embodiment, the metal wires on the edge of the wipe 100 are fused or sealed, using a high-pressure process. The high-energy process or the high-pressure process reduces or eliminates fraying of the edges of the wipe 100.

In some embodiments, the wipe 100 includes a textured surface. The surface of the mesh 1 and/or metal wire may be physically modified to impart a textured surface (visible or microscopic) to enhance collection of samples from surfaces. Surface modification may include, but is not limited to, twisting, abrading or breaking the metal wires to create a suede-like texture on the wipe 100.

In other embodiments, the wipe 100 includes a coating. The surface of the mesh 1 and/or metal wire may be plated with other materials, such as other metals, for example, gold, for specific properties, such that the wipe is chemically inert. The surface of the mesh 1 and/or metal wire may be coated with chemicals for specific properties, such as phosphates for chemical adhesion or a chemical indicator. The coating may be applied by a process such as spraying, painting, chemical techniques, electrochemical techniques, chemical vapor deposition, physical vapor deposition, roll-to-roll coating, and the like. Other methods of applying coatings will be recognized by those skilled in the art.

While FIG. 3 shows the wipe 100 formed from a mesh 1, other structures may be used to create the wipe 100. In an embodiment, the wipe 100 is formed from a mesh 1 as described above to provide a large surface area for adsorption. In an embodiment, the wipe 100 is formed from a perforated sheet material such as a perforated metal foil. Perforated foil material, which is a product that is made from sheet that has been fed through a machine that forms holes in the sheet, may also be used. In yet another embodiment, the wipe 100 is formed from an expanded metal sheet that is made by first creating multiple slits in the sheet, and then stretching the sheet. The perforated metal sheet and expanded metal sheet may require accurate machining with tight tolerances.

FIG. 4 is a flowchart of an example of a process 200 for manufacturing the wipe 100 (e.g., the variety of wipes 100A-D). Various steps described herein with respect to the process 200 are capable of being executed simultaneously, in parallel, or in an order that differs from the illustrated serial manner of execution. The process 200 may also be capable of being executed using fewer steps than are shown in the illustrated embodiment.

The process 200 is used to convert a sheet of metal into the wipe 100. At step 201, the wipe 100 is cut from a sheet of metal. The metal is, for example, copper, aluminum, stainless steel, a combination of these metals, or a metal/alloy that will be recognized by those skilled in the art. The sheet of metal is preferably a mesh 1 of wire metal. In other embodiments, the sheet of metal is, for example, a perforated metal foil, such as a perforated metal sheet and an expanded metal sheet (foil) or the like. The cutting of step 201 can be performed by die cutting, laser cutting, or waterjet cutting. Other suitable methods of cutting metal are known to those skilled in the art.

The wipe 100 may be cut into a variety of shapes. FIGS. 2A-2D illustrate an irregular polygon, but one skilled in the art will appreciate that other shapes are possible, such as, but not limited to, rectangles, ovals and the like.

In one embodiment, at step 202, at least one aperture 102 is cut into the wipe 100. The at least one aperture 102 allows for attaching the wipe 100 to a collection tool. The at least one aperture 102 may also be used to secure the wipe 100 to the detector 10. Similar to the shape of the wipe 100, a variety of shapes are possible for the at least one aperture 102. For wipes 100 to be used with detectors 10 or tools that do not require apertures, step 202 may be omitted. In another embodiment, the wipe 100 is configured to attach to a collection tool or the detector 10 without an aperture. For example, a portion of the wipe 100 can be clasped between components of a collection tool or the detector 10.

At step 204, edges of the wipe 100, formed by cutting the wipe 100 from the sheet of mesh 1 at step 201 or cutting the at least one aperture 102 from the wipe 100 at step 202, may be treated to reduce or eliminate fraying. In some embodiments, the metal wires on the edge of the wipe 100 are fused or sealed as at step 204 using a high-energy process or high-pressure process to reduce or eliminate fraying of the edges of the wipe 100.

The wipe 100 is cleaned at step 205. Cleaning removes organic materials and contaminants so that only adsorbed material from surfaces are desorbed and analyzed by the detector 10. In one embodiment, the wipe 100 is cleaned by soaking the wipe 100 in, for example, an acid, which may generate waste. In another embodiment, the wipe 100 is cleaned by soaking the wipe 100 in, for example, a solvent, such as acetone, which may also generate waste.

Additionally or alternatively, the wipe 100 is cleaned at step 205 by rapidly heating the wipe 100 to a temperature greater than about 200° C. and held at the temperature for a period greater than about 10 minutes. In one embodiment, the wipe 100 is heated to a temperature greater than 100° C. for a period of greater than about 15 minutes. In another embodiment, the wipe 100 is heated to a temperature greater than about 55° C. In another embodiment, the wipe 100 is heated to a temperature greater than 400° C. The temperature is preferably higher than the temperature used by the detector 10 for desorption. In one cleaning step, the wipe 100 is held at a temperature greater than about 200° C. for a period of between about 30 minutes to 120 minutes. It will be appreciated that a variety of heating rates are possible, and that using a combination of cleaning processes, e.g., acid soaking, solvent soaking, and heating, sequentially or simultaneously may be used at step 205.

At step 209, the wipe is packaged in a clean or sterile container. A plurality of wipes 100 can be stored in the container in amounts of, for example, 25 count, 50 count, 100 count, and 200 count. The container is thoroughly cleaned to keep the wipe 100 clean until the first use to collect a sample from a surface. As described above, the wipe 100 is reusable for multiple collections of samples from surfaces. It is anticipated that wipes may be reused, about 12-40 times.

Additional processes can be carried out on the wipe 100 during process 200. In some embodiments, the wipe 100 includes a textured surface. The surface of the sheet of metal or foil (if performed prior to step 201) or the wipe 100 (if performed after step 201) may be physically modified to impart a textured surface to enhance collection of samples from surfaces at step 206. Surface modification may include, but is not limited to, twisting, abrading, breaking the metal wires and like surface modifications, to create a suede-like texture on the wipe 100.

In other embodiments, the wipe 100 includes a coating. The surface of the sheet of metal or foil (if performed prior to step 201) or the wipe 100 (if performed after step 201) may be plated with other metals for specific properties, including, but not limited to, gold for chemical inertness at step 208. The surface of the sheet of metal or foil (if performed prior to step 201) or the wipe 100 (if performed after step 201) may be coated with chemicals for specific properties, including, for example, phosphates for chemical adhesion or a chemical indicator at step 208.

In addition to using wipes 100 for collecting samples, some wipes 100 are used to calibrate a detector. For such wipes 100, an additional step of applying an explosives or narcotics trace is included in process 200. The explosives or narcotics traces is precisely measured and applied so that the accuracy of the detector 10 can be tested, and the detector 10 may be calibrated, if needed.

In some embodiments the substrate is made from a heat-tolerant non-metal material. For example, the substrate can be formed from high-heat-tolerant polymers, such as polyether ether ketone (PEEK), polyimide, silicone and like high-heat-tolerant materials. In one such embodiment, the wipe is formed as a sheet and cut into appropriately sized and shaped wipes, such as those illustrated in FIG. 2. The substrate can be formed as a mesh or it can be formed as a sheet with the pores or openings formed (as by punching, cutting or the like) to provide an appropriate porosity.

In still other embodiments, the wipes can be formed as a laminate. For example, the wipes can be formed as a laminate of copper foil and PEEK or copper foil and silicone. These laminates have the beneficial properties of metals, that is they quickly and evenly heat up and cool down, they are heat-tolerant and they dissipate heat evenly. It has been found that these laminates retain the ability to collect, e.g., adsorb, contaminants, for example, explosives and narcotics, and release, e.g., desorb, the contaminants well when subjected to heat. In addition, these laminates function well in temperatures of about 240 deg. C. and as high as 300 deg. C., making them well suited for use in known detectors. As with the previous embodiments, the substrate can be formed as a mesh or it can be formed as a sheet, for example a foil, with the pores or openings formed (as by punching, cutting or the like) to provide an appropriate porosity.

It has also been found that the laminates having an outer layer of polymer are generally chemically inert and do not catalyze the contaminants. As such, the detectors function well and appropriately, and without interference or false readings (false positives or false negatives) due to reactions (chemical or otherwise) from metal/contaminant interactions.

It will also be appreciated that the polymer layer provides a degree of isolation of the metal from the object being sampled. Purely metal wipes may scratch softer surfaces or be irritating to a person's skin (when that person's skin is being sampled). The polymer coating is “softer” or less abrasive than metal, and reduces the opportunity to scratch surfaces or irritate skin.

Those skilled in the art will appreciate that the polymer coating can be applied to one side or both sides of a metal substrate to provide the beneficial characteristics noted above. Such a one sided laminate provides all of the above-noted advantages of metal, such as quick and even heat up and cool down, heat-tolerance, even heat dissipation and the ability to collect or adsorb and retain contaminants, as well as the polymer's advantages of being chemically inert (not catalyzing contaminants) and less abrasive than metal. Moreover, such a one-sided or two-sided polymer coating has the further advantage of containing the edges of a metal substrate, thus preventing fraying of the edges or other damage to the underlying metal.

Thus, devices and methods consistent with the present disclosure provide, among other things, a wipe for collecting a sample from a surface for analysis in a detector, and method of manufacturing the wipe. In some embodiments, the wipe is formed from a mesh made of a metal wire, such as copper, aluminum, and stainless steel, a combination of these metals, or another metal/alloy that will be recognized by those skilled in the art. In some embodiment, the wipe is formed from non-metal materials, such as polymeric materials, such as a high-heat-tolerant polymeric material, or a laminate of polymeric materials and, for example, a metal. Suitable high-heat tolerant materials may include PEEK, polyimides, silicones and the like. The disclosure provides the benefits of more adsorption and reusability than known wipes.

In the present disclosure, unless otherwise noted, all percentages (%) are percent by weight as appropriate. In addition, the words “a” or “an” are to be taken to include both the singular and the plural. Conversely, any reference to plural items shall, where appropriate, include the singular. It will be appreciated by those skilled in the art that the relative directional terms such as upper, lower, rearward, forward and the like are for explanatory purposes only and are not intended to limit the scope of the disclosure.

All patents or patent applications referred to herein, are hereby incorporated herein by reference, whether or not specifically done so within the text of this disclosure.

From the foregoing it will be observed that numerous modification and variations can be effectuated without departing from the true spirit and scope of the novel concepts of the present film. It is to be understood that no limitation with respect to the specific embodiments illustrated is intended or should be inferred. The disclosure is intended to cover by the appended claims all such modifications as fall within the scope of the claims.

1. A wipe for collecting a sample from a surface for analysis in a detector, the wipe comprising:
a mesh made of a metal wire;
wherein the mesh is flexible and conforms to the surface for collecting the sample;
wherein the mesh adsorbs the sample from the surface;
wherein the mesh desorbs the sample in the detector; and
wherein the wipe is reusable for collecting a plurality of samples.

2. The wipe of claim 1, wherein the mesh is selected from the group consisting of a plain weave, a Dutch weave, a twilled weave, a twilled Dutch weave, and a reverse Dutch weave of the metal wire.

3. The wipe of claim 2, wherein a count of the mesh is selected from the group consisting of 200 per inch, 400 per inch, and 400×1400 per square inch.

4. The wipe of claim 1, wherein a diameter of the metal wire is selected from the group consisting of 0.001″, 0.0016″, 0.002″, 0.0021″, and 0.0028″.

5. The wipe of claim 1, further comprising a textured surface of the metal wire, wherein the textured surface is created by twisting, abrading, or breaking the metal wire.

6. The wipe of claim 1, further comprising a coating on the metal wire, wherein the coating is selected from the group consisting of gold, phosphates, and chemical indicators.

7. The wipe of claim 1, wherein the mesh adsorbs the sample from the surface at a first temperature range, and wherein the mesh desorbs the sample in the detector at a second temperature greater than the first temperature range.

8. The wipe of claim 7, wherein the first temperature range is greater than −10° C. and less than 55° C., and wherein the second temperature is greater than 100° C.

9. The wipe of claim 1, further configured to attach to a collection tool or detector.

10. A wipe for collecting a sample from a surface for analysis in a detector, the wipe comprising:
a substrate having first and second surfaces;
a high-heat tolerant polymeric material on the substrate;
a plurality of openings formed in the substrate and polymeric material to form the wipe,
wherein the wipe is flexible and conforms to the surface for collecting the sample, wherein the wipe adsorbs the sample from the surface, and desorbs the sample in the detector; and
wherein the wipe is reusable for collecting a subsequent sample.

11. The wipe of claim 10, wherein the substrate is a metal.

12. The wipe of claim 10, wherein the substrate is a polymeric material.

13. The wipe of claim 10, wherein the polymeric material is one or a combination of polyether ether ketone (PEEK), polyimide and silicone.

14. The wipe of claim 10, wherein the substrate is formed as a perforated foil and the polymeric material is applied to the perforated foil.

15. The wipe of claim 10, wherein the polymeric material is present on one of the first and second sides.

16. The wipe of claim 15, wherein the polymeric material is present one the first and second sides.

17. A method for manufacturing a wipe for collecting a sample, the method comprising the steps of:
cutting the wipe from a sheet of material, wherein the wipe is flexible and conforms to a surface for collecting the sample;
cleaning the wipe to remove contamination; and
packaging the wipe in a clean container.

18. The method of claim 17 including the step of providing a substrate having first and second sides and applying a high-heat tolerant polymeric material on the substrate.

19. The method of claim 18 wherein the high-heat tolerant polymeric material is applied on the first and second sides of the substrate.

20. The method of claim 17, wherein the substrate is formed from a metal.

21. The method of claim 17, further comprising cleaning the wipe by heating the wipe to a second temperature greater than an adsorption temperature range of the wipe for a period greater than 10 minutes.

22. The method of claim 21, wherein the wipe has an adsorption temperature range of about −10° C. to about 55° C., and wherein the wipe has a desorption temperature greater than about 100° C.

23. The method of claim 17, further comprising cleaning the wipe by soaking the wipe in an acid or a solvent.

24. The method of claim 20, wherein the metal is a sheet of metal formed as a metal wire mesh, and wherein the mesh is formed from one or more of a plain weave, a Dutch weave, a twilled weave, a twilled Dutch weave, and a reverse Dutch weave.

25. The method of claim 20, wherein the metal is a perforated metal foil or an expanded metal sheet or foil.

26. The method of claim 24, further comprising texturing a surface of the wipe by twisting, abrading, or breaking the metal wire.

27. The method of claim 17, further comprising applying a coating to the wipe, wherein the coating is one or more of gold, phosphates, and chemical indicators.

28. The method of claim 24, further comprising fusing edges of the wipe using a high-energy or high-pressure process to reduce fraying of edges of the wipe.

29. The method of claim 17, further comprising applying an explosives or narcotics trace, wherein the wipe is used for calibrating a detector.

30. A method for collecting a sample from a surface using a wipe, the method comprising the steps of:
contacting the wipe to a surface, wherein the wipe is formed as a perforated coated substrate, is flexible, conforms to the surface, and adsorbs the sample from the surface;
placing the wipe into a detector and heating the wipe to desorb the sample for analysis; and
reusing the wipe for collecting one or more subsequent samples.
